(19) United States
(12) Patent Application Publication
(10) Pub. No.: US 2010/0255110 A1
(43) Pub. Date: Oct. 7, 2010

(54) FUNCTIONAL AGRICULTURAL PRODUCT AND METHOD FOR CULTIVATING THE SAME

(76) Inventors: Eui-Sik Yoon, Seoul (KR); Jong-Suk Lee, Seoul (KR)

Correspondence Address: OSTROLENK FABER GERB & SOFFEN, 1180 AVENUE OF THE AMERICAS, NEW YORK, NY, 100368403, US

(21) Appl. No.: 12/743,648

(22) PCT Filed: Dec. 5, 2008
(86) PCT No.: PCT/KR08/07227

§ 371 Date: May 19, 2010

(30) Foreign Application Priority Data
Dec. 5, 2007 (KR) 10-2007-0125405

(51) Int. Cl. A61K 9/14; A61K 33/38; A61K 33/24; A61P 25/28; A61P 39/00; A61P 9/00; A61P 35/00; A01G 1/00

(52) U.S. Cl. 424/498; 424/489; 424/618; 424/649; 47/58.1 R; 977/915

(57) ABSTRACT

Disclosed is a functional agricultural product and a method for cultivating the same. The functional agricultural product is characterized in that metallic nano particles are absorbed into the agricultural products in a particle form. Also, in the method for cultivating the functional agricultural products, a metallic nano particle colloid, in which the metallic nano particles are dispersed in water or an electrolyte, is supplied into the agricultural products. Hence, the agricultural products containing metals hard to be ionized can be cultivated by low cost.

### TECHNICAL FIELD

The present invention relates to a functional agricultural product and a method for cultivating the same, and more particularly, a functional agricultural product containing a metal, such as platinum, gold and/or silver well known as having various efficacies good to human bodies, and a method for cultivating the same.

### BACKGROUND ART

Platinum, gold and silver have been used to cure a variety of diseases. Gold ashes have been used for several clinical diseases in India since the 8^{th} century. Ayurvedic physicians have used gold ashes for cure of bronchial asthma, rheumatic arthritis, diabetes and neuropathy.

In recent time, it has been researched that a colloidal gold allows an acquisition of antibodies with a fast and high efficiency as compared to a standard scheme for raising an immunity composed of antigen and adjuvant. It can thusly be expected in view of such effect that gold can be applied to improve immunity, increase a concentration of lysozyme in blood, advance an activity of a protein complementation system, and improve immunity such as an activity of disinfection. It has been checked that the result of injecting golden colloid together with hepten or complete antigen into a laboratory animal, such as a rabbit or mouse, can induce the creation of antibodies having high activities. Also, results of a research have been published that a constant internal application has an effect on the cure of rheumatic arthritis. It has recently been found out that the colloidal gold acts on failure of memory or eyesight, sterility, an early aging process, enervation and the like, and thusly it is being added in a variety of foods or drugs for use.

Platinum has actively been studied in recent time, and used as an anti-cancer drug due to an ionic characteristic of a surface of a unique particle. From the results of the Japanese research, the usability of the platinum ultrafine particles in the cure of cardiovascular disease due to a metabolism syndrome has been found out from a test using model mice. Based upon the paper published in the 21^{st} International Society of Hypertension (ISH2006) opened in Fukuoka city, the research team of kidney and endocrine internal department in the hospital of the University of Tokyo checked that the platinum ultrafine particles removed a reactive oxygen species (ROS) by catalysis in the in vitro test. The research team fed an angiotensin II acting on raising a blood pressure and a highly salty meal to an obese model mouse (db/db mouse) to cause hyperpiesia, thereby preparing a model of a metabolism syndrome. Such model was fed with platinum nano particles with water everyday, and changes happened in the model were observed for 4 weeks. As a result, although the systolic pressure of a normal mouse (n=6) was 113 mmHg, the systolic pressure of a model mouse (n=6) without the platinum nano particle injected was raised up to 136 mmHg and that of a model mouse (n=6) having the platinum nano particle injected was decreased down to 129 mmHg. The cholesterol level of the normal mouse was 150 mg/dL and that of the non-injected mouse was 151 mg/dL, namely, they are almost the same. However, the cholesterol level of the platinum nano particle injected mouse was reduced to 140 mg/dL. In addition, it was checked that the amount of 8-iso-PGF2α in urine as a marker of oxidative stress was remarkably reduced by the injection of the platinum nano particles and a coronary fibrosis was histologically reduced by 50%. The researchers derived a conclusion from such research results that the platinum ultrafine particles were antioxidants which removed the active oxygen causing organic injuries.

As such, research results that gold, platinum, silver and the like can effectively be applied to medial fields are pouring out. Methods for injecting such substances into human bodies, as being widely used in Japan or the like, may include a method in which such metal is processed to be finely powdered, such as gilt, and added into foods to be directly taken in, a method in which the metal is processed to become nano particles so as to be colloidal and such colloidal particles are rubbed on a skin or drunk or injected into a body, a method in which to such colloid of the metallic nano particles are mixed with other medical supplies or foods to be applied into bodies, and the like.

### DISCLOSURE

### Technical Solution

However, the aforesaid methods of directly applying metals into human bodies or making such metals directly taken are not widely popularized, still causing a feeling of rejection from ordinary persons.

Accordingly, many attempts have been carried out to make such metals contained in agricultural products we are ingesting.

If it is possible to allow agricultural products to contain noble metals, particularly, the aforementioned gold, silver, platinum and the like, which are well known as having various efficacies, such agricultural products with the noble metals can easily be approached and ingested, and also the efficacies of the noble metals are added to each known efficacy of various agricultural products to expect a synergy effect therefrom. In addition, it will be obvious that a cultivation of such agricultural products containing the noble metals can derive high gains.

However, there is a problem that because such gold, silver and platinum are hard to be ionized, even if such metals are applied into soil for cultivating agricultural products, it is impossible for roots of such agricultural products to absorb such metallic components together with water.

A plant absorbs water and inorganic substances through its roots. The water is absorbed by an osmotic pressure and the inorganic substances are absorbed by an active transport of the plant as much as necessary. The active transport denotes the mediated process of moving particles against a concentration gradient by using (adenocine triphosphate) ATP type energy within lives. The inorganic substances absorbed by the plant may include iron, potassium, calcium, phosphorus, sulfur, magnesium, manganese, zinc and the like, for example. Such substances are commonly highly soluble in water and absorbed with water, thereafter being selectively actively transported by the plant. However, as mentioned above, metallic components which are hardly ionized cannot easily be absorbed with water, whereby it has been known that agricultural products containing such noble metals do not exist in nature.

As an effort to make plants contain noble metallic components, in order to extract gold using plants, C. Anderson in the Massay university in New Zealand in 1997 distributed chemicals, such as a strong acid, over an abandoned gold mine such that gold could be melt, and thereafter planted cabbage, cauliflower and turnip to absorb such gold. As a result, plants within 1 hectare absorbed 1 kg of gold. The point of Anderson's observation was to ionize gold using chemicals to allow plants to absorb such ionized gold together with water. However, he missed that the plants might be dead due to toxicity of the chemicals used for the ionization. It has widely been known that gold is ionized only by aqua regia. With Anderson's method, the gold ionized by the strong acid was a little possible to be absorbed by the plants, but the plants were not normally grown due to the toxicity of the strong acid. As a result, the metallic component absorbed by the plants could not be ingested into human bodies together with the plants.

In the meantime, a method for making plants contain silver was proposed in Korean Patent Registration No. 10-0533252, which discloses a method in which silver ion chelates to chitosan oligosaccharide ligand such that the silver could be absorbed together with the chitosan oligosaccharide. Unlike the aforementioned method using the strong acid, this method allowed plants to be a little grown into the state of being ingestible into human bodies. However, such method required complicated processes to thereby increase the production cost of agricultural products. Furthermore, silver nitrate solution was used to fabricate a processing solution, and the nitrate containing toxicity problematically remained after chelating the silver ion to the chitosan oligosaccharide. In addition, the thing which is contained in the agricultural products is not the silver itself, but the silver ion chelating to the chitosan oligosaccharide. Accordingly, upon actually ingesting the agricultural products, the silver ion with a high activity chemically reacted with other materials to be absorbed in a compound state, whereby the efficacy or property of the silver itself could not be exhibited as it was.

The present invention is derived from the recognition of the drawbacks of the method for cultivating functional agricultural products containing metals according to the related art. In order to solve such drawbacks, one object of the present invention is to provide functional agricultural products containing metals, in which a noble metal can be contained in agricultural products as it is, not in an active ionic state, to thereby enable an effective exhibition of efficacy and property of the noble metal contained when it is ingested into human bodies, and a method for cultivating the same.

Another object of the present invention is to allow a production of agricultural products containing noble metals with low price as well as an easy application in farmhouses.

In order to achieve such objects of the present invention, there is provided a functional agricultural product characterized in that metallic nano particles are absorbed in a particle form in agricultural products.

Here, preferably, each of the metallic nano particles may have a size equal to or smaller than 10 nm.

The metallic nano particles may be coated with polysorbate on its surface.

In another aspect of the present invention, there is provided a method for cultivating a functional agricultural product characterized in that a metallic nano particle colloid, in which metallic nano particles are dispersed in water or electrolyte, is supplied into agricultural products.

Here, the metallic nano particle colloid may be obtained by adding a is current to a pair of metallic electrode rods disposed to face each other in an electrolytic aqueous solution (electrolyte), in which a metallic salt is melted and polysorbate is added, with stirring the electrolyte, thereby reducing a metallic ion ionized from the metallic electrode rods in the electrolytic aqueous solution to precipitate metallic nano particles, and coating each outside of the precipitated metallic nano particles with the polysorbate.

### ADVANTAGEOUS EFFECTS

In the present invention, platinum, gold and/or silver colloids are supplied into agricultural products, including vegetables, mushrooms, medical herbs, fruit, tea leaves, grains, ginsengs and the like, for example, to be absorbed and stored therein, whereby value-added agricultural products with efficacies of the platinum, gold and/or silver can be cultivated.

In addition, the present invention allows an easy application of such cultivating method to farmhouses and also the low-cost production of agricultural products containing noble metals, resulting in enabling the practical use of functional agricultural products containing noble metals which are hard to be ionized.

Also, such agricultural products cultivated by the present invention can be used to manufacture associated products having efficacies of the platinum, gold and/or silver, such as extracts, teas, beverages and the like. Hence, the products containing the noble metals can be taken without feeling unpleasant. In addition, upon cultivating plants having a medical action, such as medical herbs, according to the present invention, products with more enhanced effects of medicine can be fabricated.

### DESCRIPTION OF DRAWINGS

FIG. 1 is a perspective view showing a root of a plant which is partially cut off to show the inside of the root, which shows a path through which the plant absorbs a nutriment;

FIG. 2 is a view showing a particle size distribution of a sample 1 analyzed by a laser particle size analyzer;

FIG. 3 is a view showing a particle size distribution of a sample 2 analyzed by a laser particle size analyzer; and

FIG. 4 is a view showing a particle size distribution of a sample 3 analyzed by a laser particle size analyzer.

### MODE FOR INVENTION

Unlike the related art method of ionizing metals to thereafter be absorbed into plants, the present invention has been derived from the conception that non-ionized metallic ultrafine particles, in detail, metallic nano particles (hereinafter, referred to as ‘metallic nano particles’) might be absorbed into plants in a particle form if the colloid of the metallic nano particles is used, and long-term tests supporting the conception. That is, the present invention provides functional agricultural products in which metallic nano particles are absorbed in a particle form.

Here, the metallic nano particles may be at least one selected from a group including gold, platinum and silver. However, the present invention may not be limited to it.

The agricultural products may be one selected from a group including vegetable, mushroom, medical herb, fruit, tea leaf, grain, ginseng and the like; however, the present invention may not be limited to such group.

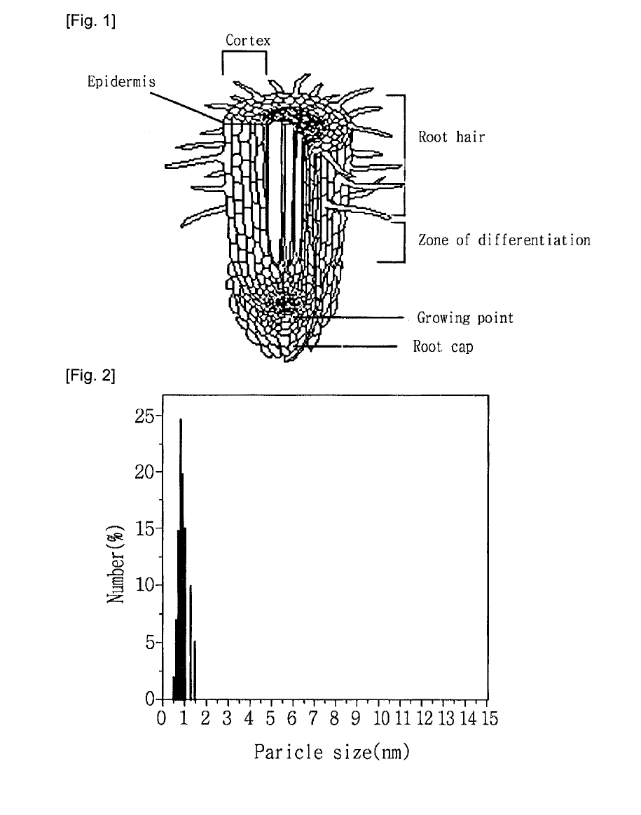

Referring to FIG. 1 which shows a path through which a plant absorbs its nutriment, various components required for the plant are absorbed into the plant together with water by an osmotic pressure. That is, water is absorbed through a path of root hairs→epidermis→cortex→endodermis→vessel by the osmotic pressure. The water flows along a cytoplasm of a root cell and a hole of a cell wall. When the water reaches the cortex before flowing into a central portion, the water flows through the cytoplasm of an endodermis tissue due to wax portions called casparian strips, which exist at the cell walls of the endodermis. Such water then moves along the cell walls to reach the vessel. Due to such complicated cellular structure, water and ionized inorganic substances are passed through but substances, such as carbohydrate or glucose with a great molecular weight, cannot be passed through.

The present inventor has developed a method for fabricating a fine stable metallic nano particle colloid and disclosed the same in Korean Patent Application No. 10-2007-0025423. Going side by side of the development, the present inventor has studied how to multilaterally utilize the developed technique. The present inventor paid attention to the results from the study on the principle and path of nutriment absorption of a plant. As a result, the present inventor recognized that since metals are stably dispersed in water, such metals may be absorbed up to the vessel of the plant together with water, and additionally, if a condition that particles have sizes as small as being capable of passing through the cell walls is met, such particles may be absorbed and contained in the plant even if it is not ionized. The metallic nano particle colloid developed by the present inventor is in a colloidal state that metallic nano particles each having a size equal to or smaller than 10 nm are evenly dispersed in water or electrolyte. The present invention has carried out many tests (experiments) based upon that such condition can be met. As a result, the present inventor has checked that when the metallic nano particle colloid is supplied into agricultural products, even a metal hard to be ionized can effectively be absorbed and contained in the agricultural products, such that the metallic components absorbed into plants can be taken into human bodies together with the plants.

In order to supply the metallic nano particle colloid to plants, substantially considering absorption efficiency, work convenience, personnel expenses and the like, a method for supplying the colloid into roots is determined to be the most practical for various farm products, and thus experiments were carried out based upon such method. Alternatively, when injecting the metallic nano particle colloid into agricultural products (e.g., using a vessel injection) or directly using the same as a solution for water culture according to the characteristic of each farm product, it was noticed that the absorption efficiency was improved to some degree. Also, when directly scattering the metallic nano particle colloid onto edible parts, such as fruit of agricultural products (e.g., for a foliar spray), it was noticed that the absorption is also possible to some degree.

In several examples of the present invention, a metallic nano particle colloid was supplied into plants to cultivate a representative agricultural products and the content of the metallic nano particles was measured to thereby determine whether the cultivation succeeded. Also, the contents were compared according to the size of each particle, to examine how the size of the particle affects the absorption of plants. The method for supplying the colloid into roots was adapted to examine whether plants absorb the metallic nano particles under the most difficult condition.

As mentioned above, in the method of fabricating the colloid of the metallic nano particles proposed by the present inventor in the prior application (Korean Patent Application No. 10-2007-0025423), a current is applied to a pair of metallic electrode rods disposed to face each other in an electrolyte, in which a metallic salt is melted and polysorbate is added, while stirring the electrolyte. Accordingly, a metallic ion is reduced in the electrolyte to precipitate metallic nano particles, and each outside of the precipitated metallic nano particles is coated with the polysorbate, to thereby prevent the sedimentation of the metallic nano particles. Alkali metallic salts may preferably be used as the metallic salts melted in the aqueous solution. The alkali metallic salts may include sodium citrate, potassium citrate, lithium citrate, sodium acetate, potassium acetate and/or sodium ascorbin, for example. When the current is applied to the pair of metallic electrode rods, a metallic ion ionized from at least one metallic electrode rods (e.g., positive pole) then exists in the aqueous solution. On the other hand, a positive ion of the alkali metallic salt melted in the aqueous solution acquires an electron from a negative pole and transfers the electron to the metallic ion ionized from the electrode rod. As a result, the metallic ion is reduced in the electrolytic aqueous solution and accordingly, metallic nano particles are precipitated. Each surface of the thusly-reduced metallic nano particles are coated with the polysorbate, thus to obtain a metallic nano particle colloid in which the agglomeration between metallic nano particles is prevented. Such method allows the metallic nano particles to be stably dispersed in the electrolytic aqueous solution and also can prevent the metallic nano particles from being rough due to the flocculation between the metallic nano particles in spite of long-term storage, which enables the provision of the metallic nano particle colloid in which the metallic nano particles each having the size equal to or smaller than 10 nm can keep stably dispersed. The examples of the present invention exemplarily employ the metallic nano particle colloid fabricated by the aforesaid method. However, any colloid, in which nano particles each having a size equal to or smaller than a preset size (preferably, 10 nm) are dispersed, may be applied to the present invention without being limited to that fabricated by the aforesaid method.

### Example 1

While stirring an electrolytic aqueous solution (electrolyte) containing a sodium citrate melted therein by using gold as an electrode, 20V current was applied into the solution in the state of maintaining the temperature of the electrolyte below 90° C., so as to prepare a metallic nano particle colloid. Here, 0.1% polysorbate was added to prepare gold colloid with average granularity of 0.8 nm (hereinafter, referred to as ‘sample 1’) and 0.005% polysorbate was added to prepare gold colloid with an average particle size of 5 nm (hereinafter, referred to as ‘sample 2’)

In the meantime, “gold colloid 20 NM Colloidal Gold” (hereinafter, referred to as ‘sample 3’) prepared by Sigma Ltd., was used as gold colloid with a diameter equal to or greater than 10 nm.

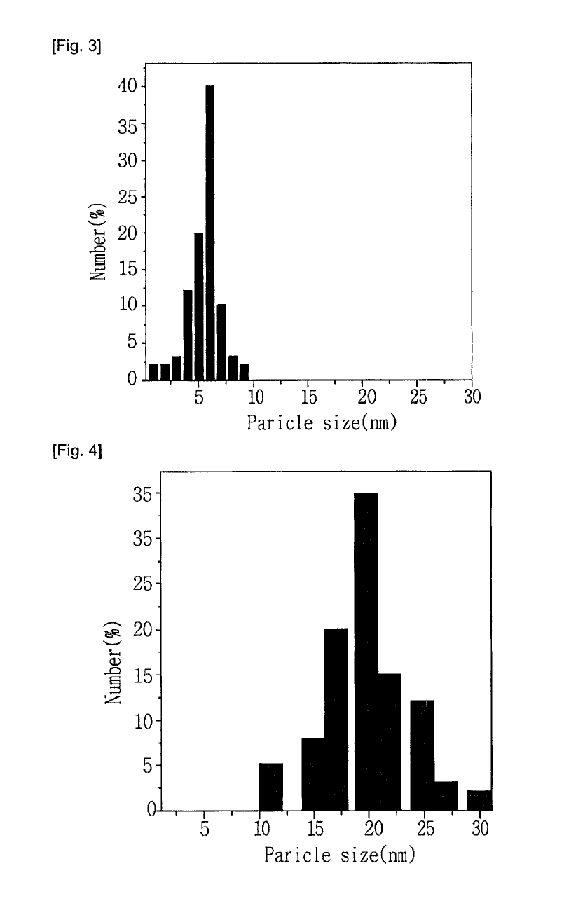

FIG. 2 shows a particle size distribution of the sample 1 analyzed by a laser particle size analyzer, in which the sample 1 has a size in the range of 0.5-2 nm. FIG. 3 shows a particle size distribution of the sample 2 analyzed by a laser particle size analyzer, in which the sample 2 has a size in the range of 1-10 nm. FIG. 4 shows a particle size distribution of the sample 3 analyzed by a laser particle size analyzer, in which the sample 3 has a size in the range of 11-30 nm.

9 pear trees were divided into three groups and then the gold colloids (concentration of 10 ppm) of the samples 1, 2 and 3 were supplied into roots of the pear trees in each group, respectively, thereby comparing and analyzing whether roots of the trees in each group absorb the corresponding gold colloid. The gold colloids were supplied twice early in August and early in September, and pears were gathered on near 20^{th} of September. Each gold colloid was evenly supplied into the roots by 5 L. The content of gold in the gathered pear was analyzed by ICP-MS.

Table 1 shows the gold contents in the pears which were grown and gathered by supplying the gold colloids according to Example 1.

TABLE 1

| Sample | Gold Content (ppm) |
|---|---|
| Sample 1 | 0.3 ± 0.02 |
| Sample 2 | 0.2 ± 0.04 |
| Sample 3 | 0 |

As shown in Table 1, it can be noticed that the gold is successfully absorbed into the pears in the samples 1 and 2 whereas the gold is not absorbed in the pears in the sample 3. Accordingly, it can be recognized that the unsuccessful absorption in the sample 3 is because a particle size is as great as being unable to pass through root cells, considering the particle size equal to or greater than 10 nm. That is, it can be reported that the gold colloid having the size equal to or smaller than 10 nm can smoothly pass through cell walls of the roots upon absorbing water by an osmotic pressure.

### Example 2

In Example 2, the gold colloid of the sample 1 was supplied into king oyster mushrooms so as to analyze whether the mushrooms absorbed the gold colloid. 10 ppm of gold nano particle colloid was prepared as shown in Example 1. When a mushroom cultivator cultivated the king oyster mushrooms, 30 ml gold colloid was injected per a bottle of mushroom at the step of a germination induction. After 15 days from the injection the mushrooms were gathered and then the gold contents of the mushrooms were analyzed.

### Example 3

In Example 3, the gold colloid of the sample 1 was supplied into cultivate green tea plants. 10 ppm of gold nano particle colloid was prepared as shown in Example 1. 3 L gold colloid was supplied per week about the middle of March and green tea leaves were gathered late in April, to analyze the gold contents by ICP-MS.

TABLE 2

| Gold Content of king oyster | Gold Content of Green |
|---|---|
| mushroom (ppm) | tea leaf (ppm) |
| 2 ± 0.4 | 0.4 ± 0.03 |

Table 2 shows the results of the ICP-MS analysis of the gold contents in the king oyster mushroom of Example 2 and the green tea leaf of Example 3. 2 ppm gold on the average was detected from the king oyster mushroom, and 0.4 ppm gold on the overage was detected from the green tea leaf. Such results can show that the mushrooms, a type of fungi, and the green tea leaf can absorb gold.

1. A functional agricultural product in which metallic nano particles are absorbed in a particle form.

2. The product of claim 1, wherein each size of the metallic nano particles is equal to or smaller than 10 nm.

3. The product of claim 1, wherein each of the metallic nano particles is coated with polysorbate on a surface thereof.

4. The product of claim 1, wherein the metallic nano particles are at least one selected from a group including gold, platinum and silver.

5. The product of claim 1, wherein the agricultural product is one selected from a group including vegetable, mushroom, medical herb, fruit, tea leaf, grain and ginseng.

6. The product of claim 1, wherein the metallic nano particle is absorbed via a vessel of the agricultural product.

7. A method for cultivating a functional agricultural product characterized in that a metallic nano particle colloid, in which metallic nano particles are dispersed in water or electrolyte, is supplied into an agricultural product.

8. The method of claim 7, wherein each size of the metallic nano particles dispersed in the metallic nano particle colloid is equal to or smaller than 10 nm.

9. The method of claim 7, wherein the metallic nano particle colloid is supplied on the soil near roots of the agricultural product so as to be absorbed by the roots.

10. The method of claim 7, wherein the metallic nano particle colloid is scattered on the agricultural product by a foliar spray.

11. The method of claim 7, wherein the metallic nano particle colloid is supplied into the agricultural product by a vessel injection.

12. The method of claim 7, wherein the metallic nano particle colloid is obtained, by adding a current to a pair of metallic electrode rods disposed to face each other in an electrolytic aqueous solution (electrolyte), in which a metallic salt is melted and polysorbate is added, with stirring the electrolyte, thereby reducing a metallic ion ionized from the metallic electrode rods in the electrolytic aqueous solution to precipitate metallic nano particles, and coating each outside of the precipitated metallic nano particles with the polysorbate.
